(19) United States
(12) Patent Application Publication
(10) Pub. No.: US 2025/0195789 A1
(43) Pub. Date: Jun. 19, 2025

(54) TRANSRADIAL APPROACH/ANGIOGRAPHY BOARD

(71) Applicant: THE UNIVERSITY OF CHICAGO, Chicago, IL (US)

(72) Inventors: Sandeep NATHAN, Chicago, IL (US); Anokha NATHAN, Chicago, IL (US)

(21) Appl. No.: 18/847,129

(22) PCT Filed: Mar. 17, 2023
(86) PCT No.: PCT/US2023/064634

§ 371 Date: Sep. 13, 2024

Related U.S. Application Data
(60) Provisional application No. 63/320,929, filed on Mar. 17, 2022.

(51) Int. Cl. A61M 5/52; A61M 25/01

(52) U.S. Cl. CPC A61M 5/52; A61M 25/01; A61M 2210/125

(57) ABSTRACT

The present disclosure includes an arm support for supporting a patient's arm during a procedure, such as a cardiac catheterization procedure. Some arm supports include a unitary body defining: a lower portion, an upper portion, a first end, a second end, a proximal side, and a distal side of the arm support, where the lower portion of the unitary body defines a mount configured to be coupled to a patient support, and where the upper portion of the unitary body defines a support surface extending between the first and second ends, where the support surface is shaped to support a patient's arm.

### CROSS-REFERENCE TO RELATED APPLICATIONS

This application claims the benefit of priority to U.S. Provisional Patent Application Ser. No. 63/320,929, filed Mar. 17, 2022, hereby incorporated by reference in its entirety.

### FIELD OF INVENTION

The present invention relates generally to apparatuses and methods for patient support during catheterization procedures; and more specifically, but not by way of limitation, to facilitate forearm positioning for catheter-based cardiac procedures performed using distal radial arterial approaches (e.g., during left radial approach angiography and intervention).

### BACKGROUND

Cardiac catheterization, or angiography, is a procedure used to treat and diagnose certain heart conditions. Cardiac catheterization can be performed to achieve access to patient's arterial system through the patient's femoral artery (groin), brachial artery (elbow), or radial artery in the wrist. The transfemoral, through groin, approach to performing cardiac catheterization has typically been more prevalent in invasive cardiology, but the transradial, through wrist, approach is gaining increasing popularity due to technical advances with catheters and lower complication rates than the transfemoral approach.

Despite growing interest, the transradial approach (“TRA”) is still a minority (approx. 25%) of 2-3 million overall cardiac catheterization cases annually. Hesitation to adopt TRA for the majority of cases may stem from a lack of familiarity with patient positioning, catheter manipulation, ergonomics, and other factors. There is currently no uniform approach for setting up or performing cardiac catheterization using the TRA. Setup may vary greatly even within a single hospital, based on factors like patient comfort and individual operator preferences. This inconsistency can create an inefficient working environment by frustrating staff.

A limited number of options currently exist for positioning patient arms during a TRA cardiac catheterization. One known method is to use a flat support board with the addition of towels, pillows or other items to adjust, or prop up a patients' arm. Some flat support boards are made available for commercial purchase, but a substantial number are fabricated in-house by repurposing available flat, rigid items such as slide boards, scrap acrylic, or other leftover materials found in the hospital. Other commercial support devices created for arm access can be utilized during TRA, but few, if any, are specifically designed for the challenges of a TRA cardiac catheterization. Known options still require that constant adjustments be made using repurposed supports (pillows, towels, foam inserts, etc.) which vary with each patient and operator preference.

To date, no single medical device exists to rapidly and comfortably position a patient's left forearm in an adducted fashion with either pronation or supination (contingent on the TRA). Thus, there exists a need for a reusable TRA board, suitable for high-volume TRA operators, and/or designed for both patient comfort and operator efficiency.

### SUMMARY

The present disclosure describes an arm support for supporting a patient's arm during a procedure, such as a cardiac catheterization procedure. The arm support at least includes a unitary body including a lower portion, an upper portion, a first end, a second end, a proximal side, and a distal side. The lower portion defines a mount configured to be coupled to a patient support such that the upper portion of the unitary body is supported above at least a part of the patient support. The upper portion defines a support surface configured to support the patient's arm. For example, in some implementations, the support surface extends between the first and second ends, and is shaped to support a patient's forearm with the patient's corresponding wrist in either a supinated or pronated position for patient comfort and operator efficiency. In other implementations, the support surface is shaped to extend over a portion of the patient such that the distal side is oriented upward to define the support surface to support the patient's arm in either a supinated or pronated position for patient comfort and operator efficiency. Additionally, the arm support can include a pre-formed structure or can be moveable for an individual patient and/or based on procedure

In various aspects, the apparatus is a medical arm board for supporting a patient's arm during a procedure, that is able to rapidly and ergonomically position patients' forearm in an adducted fashion with either pronation or supination. The apparatus may be unitary such that the support can be easily sanitized and reused for efficient use with high-volume TRA operators. Additionally, the apparatus can include a pre-formed structure or can be moveable for customizable support based on individualized patient and operator preferences. This disclosure includes embodiments of apparatuses, for transradial catheterization boards. At least some of the present embodiments are configured to rapidly and comfortably position a patient's left forearm during a procedure such as a TRA cardiac catheterization procedure.

Some embodiments of the present apparatuses (e.g., an arm support for supporting a patient's arm during procedures, such as a cardiac catheterization procedure) comprise: a unitary body that defines: a lower portion, an upper portion, a first end, a second end, a proximal side, and a distal side of the apparatus, where the lower portion of the unitary body defines a mount configured to be coupled to a patient support such that the upper portion of the unitary body is supported above at least a part of the patient support and the upper portion of the unitary body defines a support surface extending between the first and second ends, and where the support surface is shaped to support a patient's forearm with the patient's corresponding wrist in either a supinated or pronated position. In some embodiments, the proximal side is configured to face the patient.

In some of the foregoing embodiments of the present apparatuses, the support surface is defined by the proximal side of the unitary body. Additionally, or alternatively, the mount includes an upright portion and a lateral portion, the proximal side of the upright portion faces in a first direction and the lateral portion extending away from the upright portion in the first direction.

In some of the foregoing embodiments of the present apparatuses, the support surface includes an elbow support portion that, when the unitary body is coupled to the patient support, is lower than a first part of the support surface that is between the elbow support portion and the first end and is also lower than a second part of the support surface that is between the elbow support portion and the second end. In some such embodiments, the elbow support portion is closer to the first part than to the second part. Additionally, or alternatively, the unitary body comprises a single layer of material spanning the upper and lower portions. In some embodiments, the single layer of material comprises a polymer, while in other embodiments, the single layer of material comprises a metal.

Some embodiments of the present apparatuses comprise: a unitary body defining: a lower portion, an upper portion, a first end, and a second end; where the lower portion of the unitary body defines a mount configured to be coupled to a patient support such that the upper portion of the unitary body is supported above at least a part of the patient support; and an upper portion defining a support surface that has an elbow support portion, an upper support portion extending from the elbow support portion toward the first end of the unitary body, and a forearm support portion extending from the elbow support portion toward the second end of the unitary body. In some embodiments, when the lower portion is coupled to a patient support the forearm support portion of the support surface extends upward from at least part of the patient support as the distance from the elbow support portion increases. In some embodiments, the forearm support portion of the surface support has a greater length than the upper support portion.

In some of the foregoing embodiments of the present apparatuses, the unitary body further defines a distal side and a proximal side, where at least a portion of the proximal side is configured to face the patient and at least a portion of the distal side is configured to face away from the patient. In some embodiments, the upper support portion of the support surface extends downward toward at least part of the patient support with decreasing distance from the elbow support portion. In some embodiments, the upper support portion is substantially parallel to a patient support surface of the patient support. In some embodiments, the upper portion of the unitary body, going from bottom to top, sequentially: extends in a proximal direction over at least a portion of the patient support, bends toward a distal direction, and extends in a distal direction such that the support surface is defined by the proximal side of upper portion of the unitary body. In some embodiments, the support surface includes a wrist support portion extending from the forearm support portion in a direction toward the second end and configured to support a patient's wrist in a supinated or pronated position.

Some embodiments of the present apparatuses comprise: a unitary body defining: a lower portion, an upper portion, a first end, a second end, a proximal side, and a distal side; where the lower portion of the unitary body defines a mount configured to be coupled to a patient support such that the upper portion of the unitary body is supported above at least a portion of the patient support, and where the upper portion of the unitary body defines a support surface extending between the first and second ends, the support surface shaped to extend over a portion of the patient such that the distal side of the unitary body is oriented upward to define the support surface to support the patient's arm in either a supinated or pronated position. In some embodiments, the lower portion of the unitary body is rigid and the upper portion of the unitary body is flexible. In some embodiments, the upper portion comprises flexible carbon fiber sheets, ballistic nylon or vinyl reinforced with vinyl mesh.

In some embodiments, the apparatus has a first cross-sectional plane, a second cross-sectional plane, closer to the second end than the first cross-sectional plane; and a third cross-sectional plane between the first cross-sectional plane and the second cross-sectional plane; where a radius of curvature of the unitary body at the third cross-sectional plane is smaller than a radius of curvature of the unitary body at the second cross-sectional plane, and the radius of curvature of the unitary body at the third cross-sectional plane is greater than a radius of curvature of the unitary body at the first cross-sectional plane. In some embodiments of the apparatuses, the upper portion has a greater width at the third cross-sectional plane than at the first or second cross-sectional plane. In some embodiments, the upper portion of the unitary body has an inflection point at the third cross sectional plane. In some embodiments, a portion of the upper portion extends in substantially the same direction as a portion of the lower portion.

Some embodiments of the present apparatuses comprise a handgrip coupled to the upper portion of the unitary body; and fasteners coupled to the lower portion of the unitary body and configured to attach to the upper portion of the unitary body to secure the upper portion in a stationary position.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed configuration, the term “substantially” may be substituted with “within [a percentage] of” what is specified, where the percentage includes 0.1, 1, 5, and 10 percent.

Further, an apparatus or system that is configured in a certain way is configured in at least that way, but it can also be configured in other ways than those specifically described.

The terms “comprise” (and any form of comprise, such as “comprises” and “comprising”), “have” (and any form of have, such as “has” and “having”), and “include” (and any form of include, such as “includes” and “including”) are open-ended linking verbs. As a result, an apparatus that “comprises,” “has,” or “includes” one or more elements possesses those one or more elements, but is not limited to possessing only those elements. Likewise, a method that “comprises.” “has.” or “includes” one or more steps possesses those one or more steps, but is not limited to possessing only those one or more steps.

Any configuration of any of the apparatuses, systems, and methods can consist of or consist essentially of—rather than comprise/include/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one configuration may be applied to other configurations, even though not described or illustrated, unless expressly prohibited by this disclosure or the nature of the configurations.

Some details associated with the configurations described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the configuration depicted in the figures.

FIG. 1 is a perspective view of a distal side of a first configuration of the present apparatuses, shown in use supporting the arm of a patient.

FIGS. 2-3 are perspective views of a proximal side and distal side, respectively, of the first configuration.

FIGS. 4A-4C are cross-sectional views of a second configuration of the present apparatuses.

FIGS. 5-6 are perspective views of the proximal side of the first configuration, shown in use supporting the arm of a patient.

FIG. 7 is a perspective view of the distal side of the second configuration, shown in use supporting the arm of a patient.

FIGS. 8-9 are perspective views of a proximal side and a distal side, respectively, of the second configuration.

FIGS. 10A-10C are cross-sectional views of the first configuration.

FIGS. 11-12 are perspective views of the proximal side of a second configuration, shown in use supporting the arm of a patient.

### DETAILED DESCRIPTION

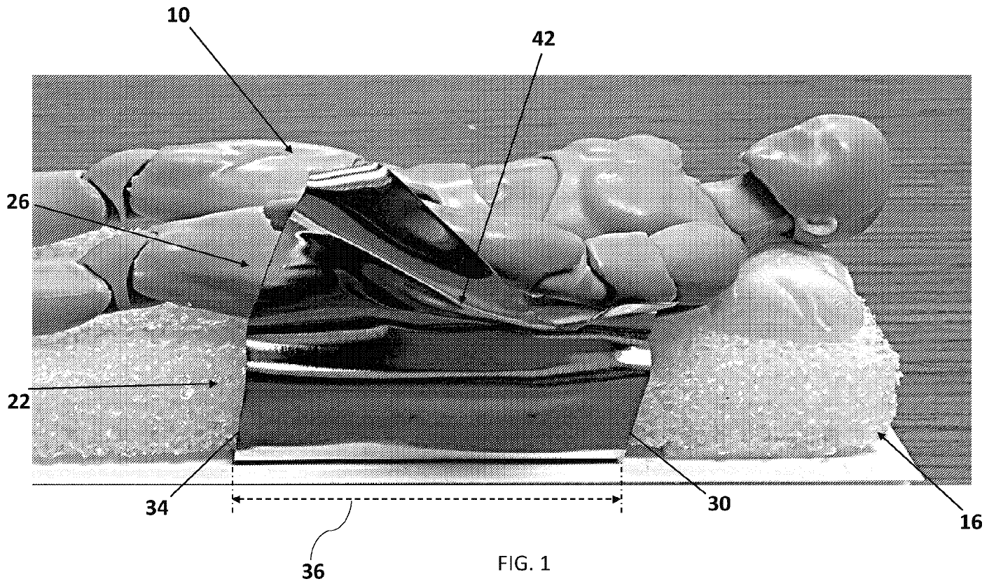

Referring now to the drawings, and more particularly to FIG. 1, shown therein and designated by the reference numeral 10 is a first configuration of the present apparatuses. In the depicted configuration, apparatus 10 is an arm support (i.e., is configured to support a patient's arm, as shown in FIG. 1). Arm support 10 may be employed to support a patient's arm during any intravenous procedure, such as, for example, during a cardiac catheterization procedure using a transradial approach (TRA). For example, in this configuration, arm support 10 is configured to support a patient arm in an ergonomic position that facilitates access of the wrist and hand of the patient while maintaining an acceptable level of comfort for the patient.

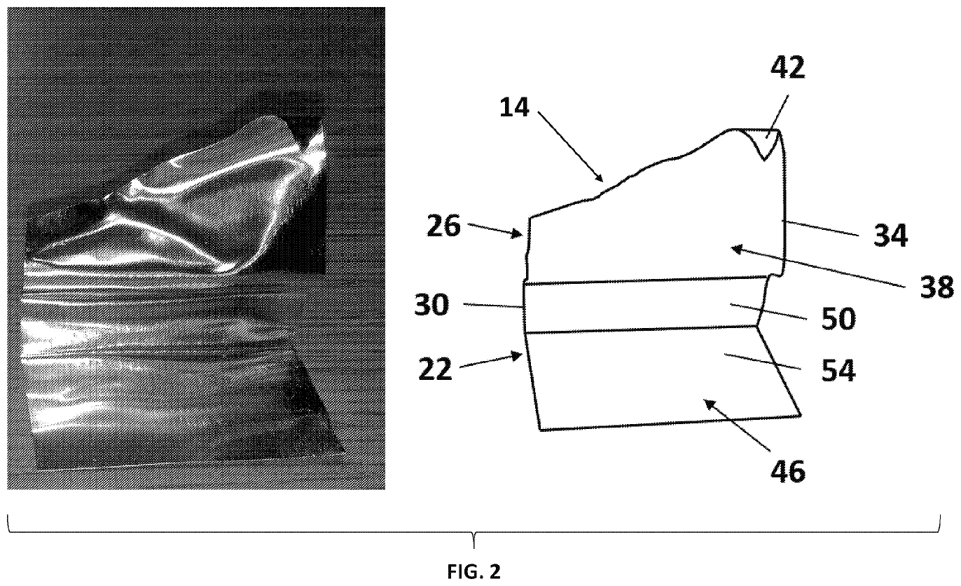

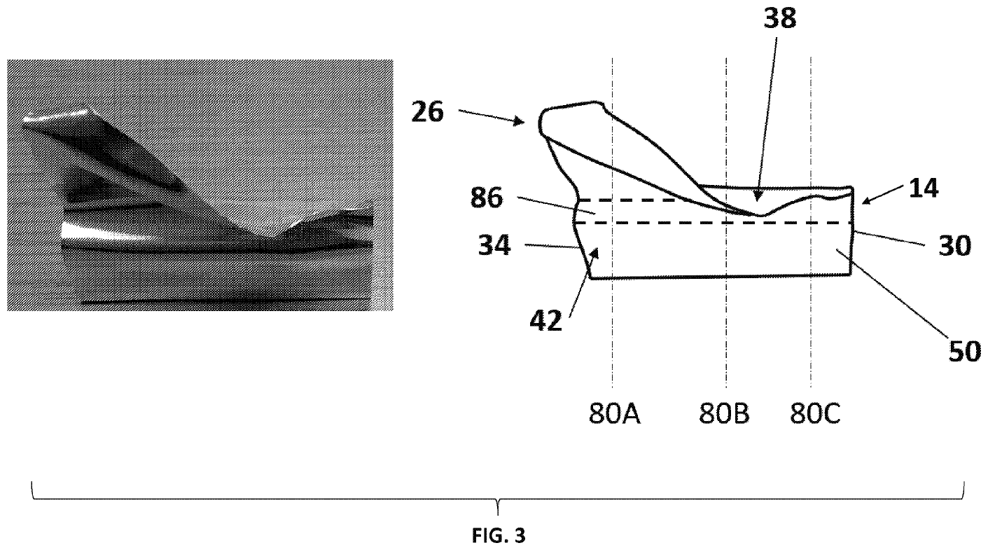

In the configuration shown in FIGS. 1-4C, arm support 10 comprises a unitary body formed of a single continuous piece of material. Arm support 10 can be shaped or formed by any of various known methods including, but not limited to, molding, thermosetting, casting, machining, pressing and/or the like. In some configurations, arm support 10 comprises any suitably rigid material including but not limited to, a metal (e.g., stainless steel, aluminum, titanium), a polymer, composite material, and/or the like.

In some configurations, arm support 10 comprises a single layer of material, while in other configurations, arm support 10 includes a material with a plurality of layers, such as, fiber-reinforced polymers, composite materials, or other material that permits the arm support 10 to function as described. In some configurations, at least a portion of arm support 10 comprises a radiolucent material that at least partially obstructs or impedes the passage of X-rays through the material, for example to assist with identifying position of the patient's arm during X-ray imaging.

In the configuration shown, support 10 comprises a one-piece unitary body 14 that defines substantially all of the support. In the depicted configuration, body 14 is configured to be coupled to patient support 16 such as, for example, a mattress, a cushioned mat, an operating table, a chair, a hospital bed, a stretcher or other suitable medium used to support a patient. In one example, patient support 16 is a mattress positioned on an operating table.

As shown, body 14 defines a lower portion 22, an upper portion 26, a first end 30, a second end 34, a proximal side 38, and a distal side 42. In this configuration, when support 10 is in use, body 14 is configured such that proximal side 38 generally faces toward the patient, and distal side 42 generally faces away from the patient. Lower portion 22 defines a mount or base 46 configured to be coupled to a patient support (e.g., 16) such that upper portion 26 is supported above at least part of the patient support 16 (e.g., above the top of the mattress for the bed depicted in FIG. 1). In the depicted configuration, a height of arm support 10 may increase as body 14 extends from first end 30 toward second end 34. In the configuration shown, a length 36 between the first end 30 and second end 34 may be between 2 feet and 3 feet. In other configurations, length 36 may be less than 2 feet or more than 3 feet, for example, equal to any one of or between any two of 1, 2, 3, or 4 ft. In the configuration shown, a portion of mount 46 is substantially planar to couple to a corresponding planar portion of patient support 16 (e.g., without requiring fasteners or additional features). However, mount 46 may be curved, non-planar or comprise clamps, straps, adhesive, or fasteners, to couple body 14 to patient support 16.

In some configurations, mount or base 46 is configured to couple body 14 to patient support 16 rigidly, and body 14 is sufficiently rigid, that upper portion 26 remains stationary despite typical forces exerted by a patient and/or operator during an intravenous procedure. For example, in the depicted configuration, mount 46 includes an upright portion 50 and a first lateral portion 54, with proximal side 38 of upright portion 50 facing in a first direction 58 and first lateral portion 54 extending away from upright portion 50 in first direction 58. In the depicted configuration, first lateral portion 54 may have a length 56 that is sufficient to stabilize support 10 from typical forces applied to support 10 during intravenous procedures (e.g., the weight of a patient's arm).

In the depicted configuration, a part of upper portion 26 extends from lower portion 22 in a distal direction to define a second lateral portion 86 to position upper portion 26 laterally inward of the outer side of patient support 14 (e.g., to improve ergonomic positioning of the patient's arm) In this configuration, a length 88 of second lateral portion 86 is less than or equal to 60% of length 56 of first lateral portion 54. In other variations, length 88 may be equal to any one of or between any two of: 30%, 40%, 50%, 60%, 70%, 80%, 90%, 100%, 110%, 120%, 130%, 140%, and/or 150% of length 56.

As shown, the upright portion has a height 60 that corresponds to a height of patient support 16. For example, in the depicted configuration height 60 is substantially equal to a thickness of a mattress (e.g., 16) to utilize frictional forces to easily couple mount 46 to patient support 16. Height 60 of upright portion 50 may be defined by the distance between first lateral portion 54 and second lateral portion 86. In the depicted configuration, height 60 is less than or equal to 30% of the maximum height of body 14. In other variations, height 60 may be equal to any one of or between any two of: 10%, 20%, 30%, 40%, 50%, 60%, or more of the maximum height of body 14.

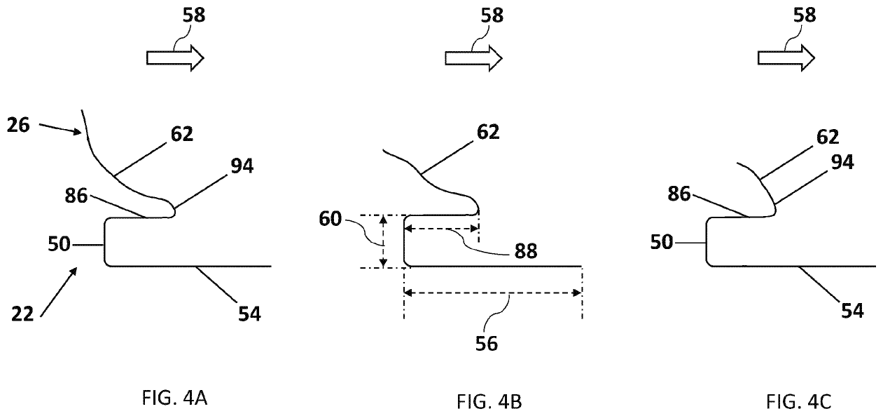

In the configuration shown, upper portion 26 may also include a bend 94. For example, in the depicted configurations in FIGS. 4A-4C, upper portion from bottom to top, sequentially: extends in a proximal direction over at least a portion of patient support 16 (e.g., second lateral portion 86), bends toward a distal direction (e.g., bend 94), and extends toward a distal direction such that support surface 62 is defined by proximal side 38. In the depicted configuration, the maximum angle of bend 94 (maximum angle at a point along length 36) is greater than or equal to 90 degrees. In this configuration, the angle of bend 94 varies but is greater than 90 degrees along the entirety of length 36. In other variations, the maximum angle of bend 94 can be equal to, or between any two of: 90, 100, 110, 120, 130, 140, 150, 160, and/or 180 degrees; and/or the angle of bend 94 can be constant along the entirety of length 36.

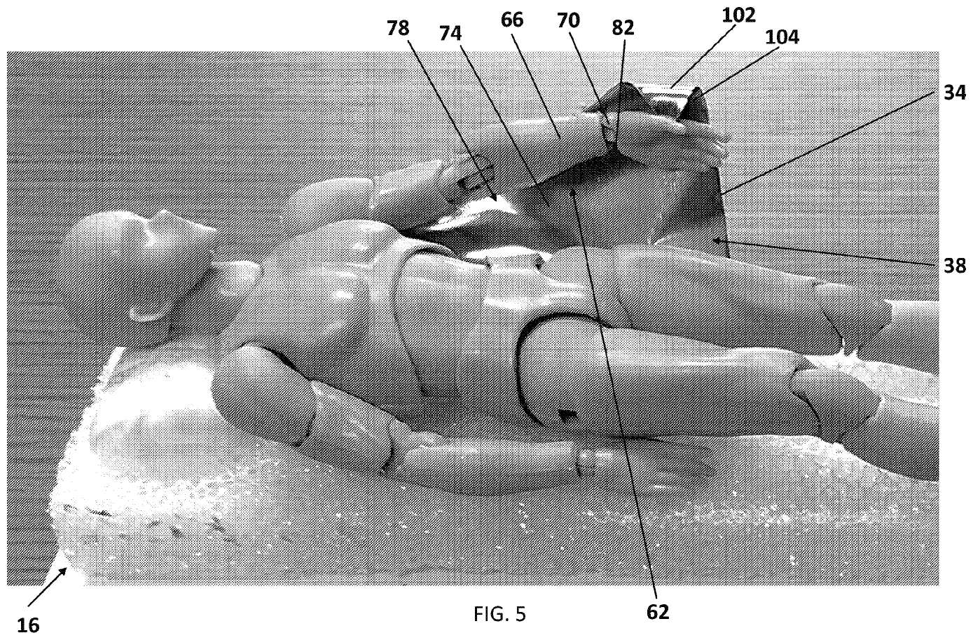

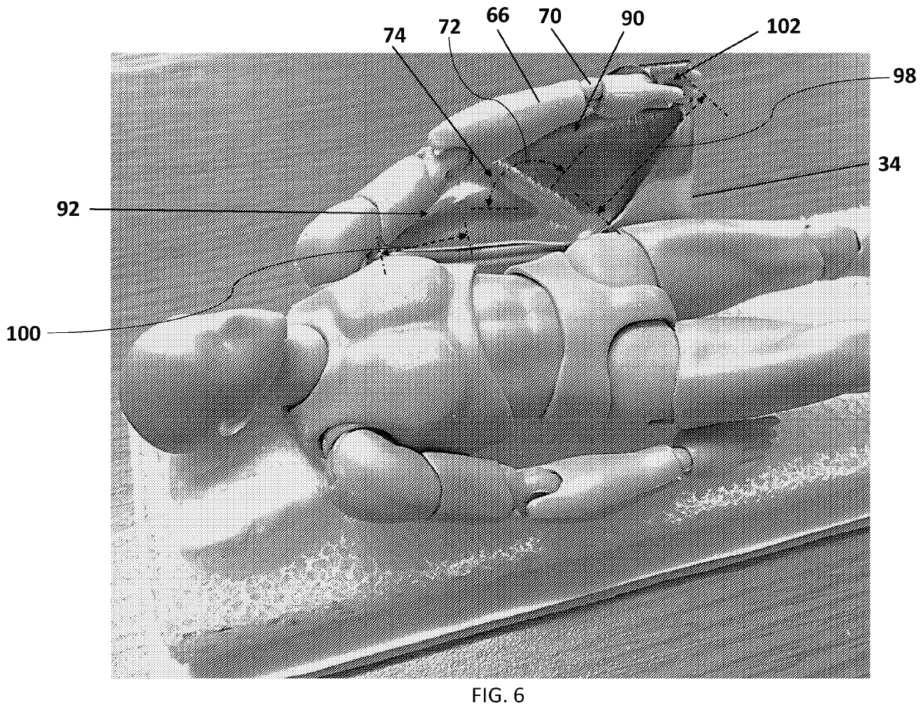

In the configuration shown, upper portion 26 defines a support surface 62 extending between first and second ends 30 and 34, and support surface 62 is shaped to support a patient's forearm 66 with the patient's corresponding wrist 70 (of the same arm as forearm 66) in an ergonomic position that facilitates access of the wrist and hand of the patient while maintaining an acceptable level of comfort for the patient. In this configuration, body 14 is formed such that proximal side 38 of body 14 defines support surface 62. In some configurations, support surface 62 may comprise a plurality of contours to allow placement of a patient's arm in a plurality of positions based on patient comfort, arm mobility, orthopedic limitations, anatomy, and size of the patient's arm. In some configurations, such as the one shown in FIGS. 5-6, support surface 62 includes an elbow support portion 74 which, when body 14 is coupled to patient support 16: (1) is lower than a first part 78 of support surface 62 that is between elbow support portion 74 and first end 30; and (2) is also lower than a second part 82 of support surface 62 that is between elbow support portion 74 and second end 34. In this configuration, elbow support portion 74 is closer to first part 78 than to second part 82. In the configuration shown, a part of elbow support portion 74 extends in a distal direction (opposite to direction 58) past upright portion 50 of lower portion 22 to accommodate elbow placement of a patients with various lengths of extremities.

In the configuration shown, support surface 62 includes an upper support portion 92 extending from elbow support portion 74 toward first end 30 and a forearm support portion 90 extending from elbow support portion 74 toward second end 34. Elbow support portion 74 may connect forearm support portion 90 to upper support portion 92. In the depicted configuration, elbow support portion 74 may comprise a first part that is angularly disposed to a second part of elbow support portion 74 by an angle 72. Angle 72 between the first part and the second part of elbow support portion 74 may be any angle that permits arm support 10 to function as described in this disclosure (e.g., the angle can be equal to, or between any two of: 90, 100, 110, 120, 130, 140, and/or 150 degrees). In the configuration shown in FIG. 6, angle 72 is less than 160 degrees; however, angle 72 can be greater than 150 degrees (e.g., equal to, or between any two of: 150, 160, 170, and/or more degrees).

In the depicted configuration, forearm support portion 90 is configured to support a patient's forearm 66 and has a length 98 that is greater than a length 100 of upper support portion 92 such that a patient's forearm 66 may be placed in a manner so to not restrict blood flow. Length 98 of forearm support portion may be greater than or equal to 110% of length 100 of upper support portion 92 to allow greater access to the patient's wrist 70. In some depicted configurations, when lower portion 22 is coupled to patient support 16, forearm support portion 90 extends upward from at least part of patient support 16 as the distance from elbow support portion 74 increases and upper support portion 92 extends downward toward at least part of patient support 16 with decreasing distance from elbow support portion 74. In other configurations, upper support portion 92 can be substantially parallel to a surface of patient support 16.

In some configurations, support surface 62 includes a wrist support portion 102 extending from forearm support portion 90 in a direction toward second end 34 and is configured to support the patient's wrist 70 along different planes of adduction (approaching the midline of the patient) with either supinated or pronated position. In the configurations shown in FIGS. 5 and 6, the wrist support portion 102 may form a U shape such that a portion of distal side 42 may support a patient's thumb and a portion of proximal side 38 may support a patient's corresponding palm. In the depicted configurations, wrist support portion 102 wraps completely around the patient's hand such that wrist support portion 102 defines a channel 104 where the patient may insert their hand so that a patient's corresponding wrist 70 may be easily manipulated from a supinated position to a pronated position, or vice versa, while the forearm 66 is still supported by support surface 62.

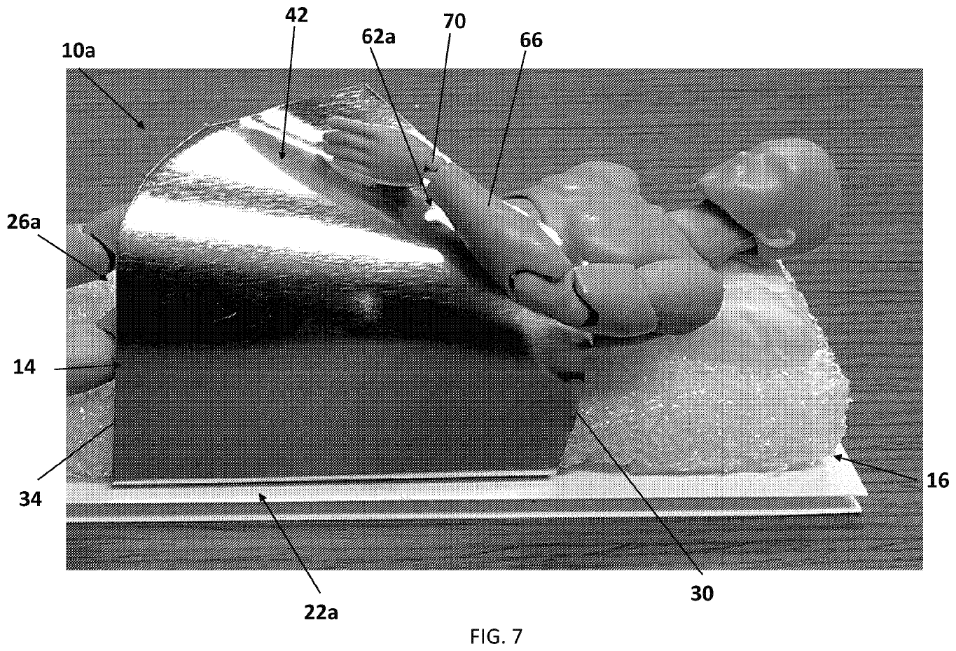

In the configuration shown in FIG. 7, shown therein and designated by the reference numeral 10a is a second configuration of the present arm support. In this configuration, components that are similar (e.g., in structure and/or function) to components discussed with reference to FIGS. 1-6 are labeled with the same reference numerals and a suffix “a.” As shown, arm support 10a may be used to stabilize a patient's arm during any intravenous procedure, such as, for example, during a cardiac catheterization procedure using a TRA.

Arm support 10a is substantially similar to arm support 10, with the primary exception that the arm support 10a protrudes over a substantial portion of the patient's body when the patient is located on patient support 16. In the configuration shown in FIGS. 7, support 10a comprises unitary body 14 defining lower portion 22a, upper portion 26a, first end 30, second end 34, proximal side 38, and distal side 42. In the configuration shown, body 14 is configured to wrap around the patient such that upper portion 26a extends over a portion of the patient and distal side 42 is oriented upward to define support surface 62a to support the patient's arm (e.g., forearm 66 or wrist 70). In this configuration, at least a portion of upper portion 26a extends in substantially the same direction as a portion of lower portion 22. As shown, upper portion 26a includes an arcuate surface with a plurality of changes in curvature.

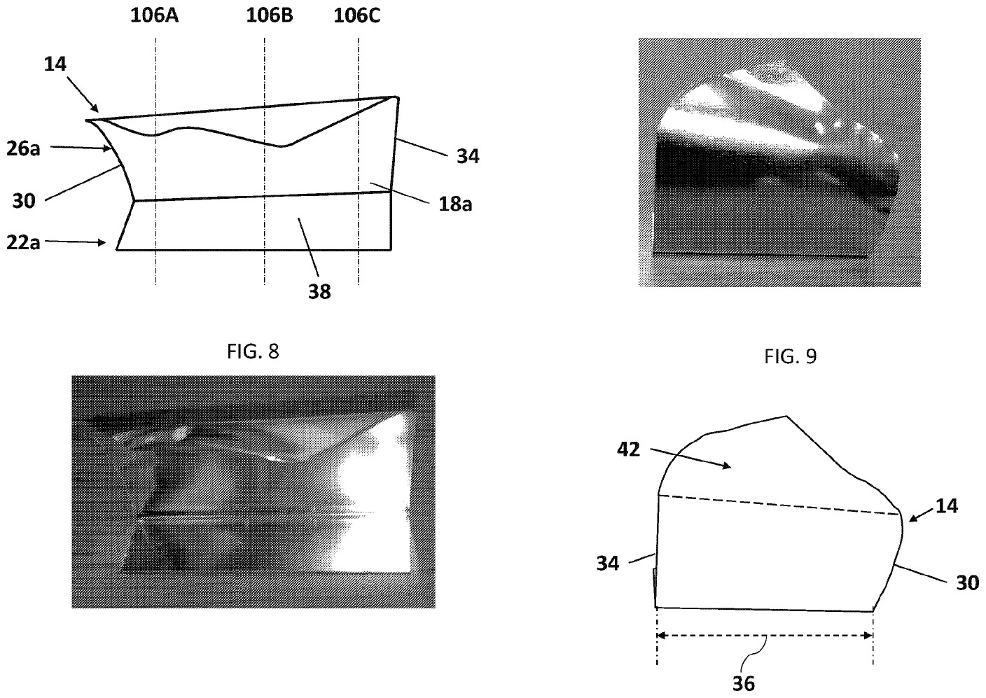

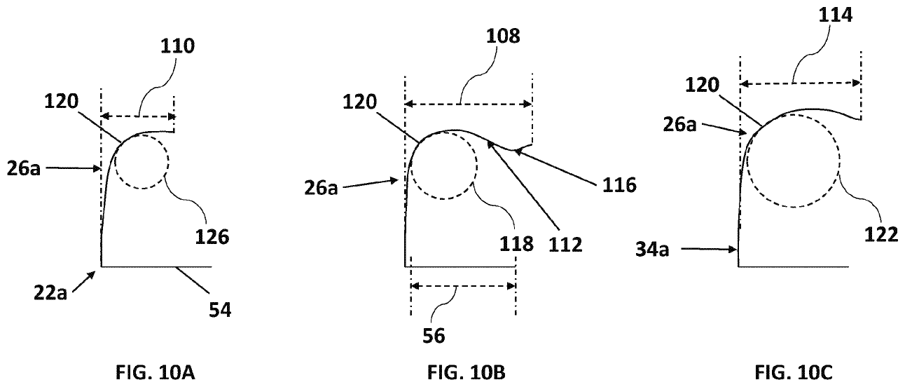

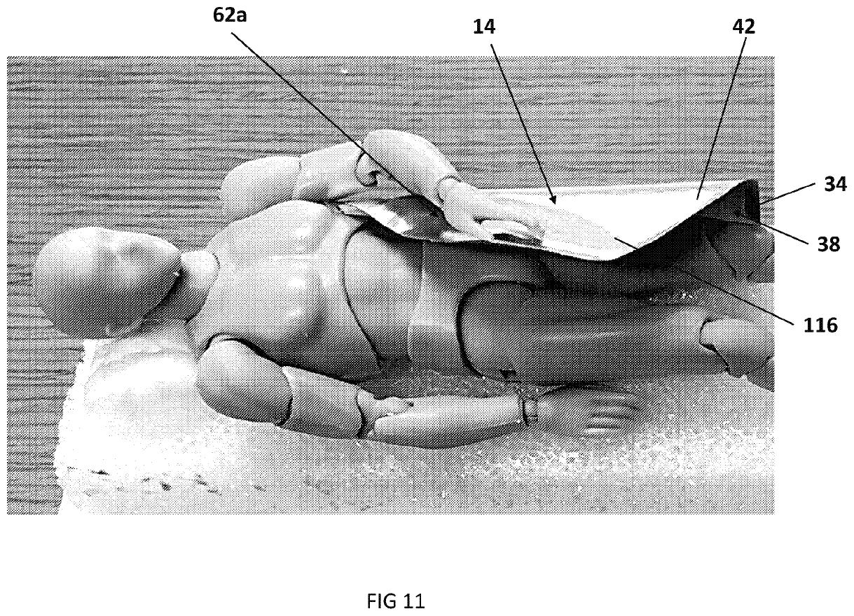

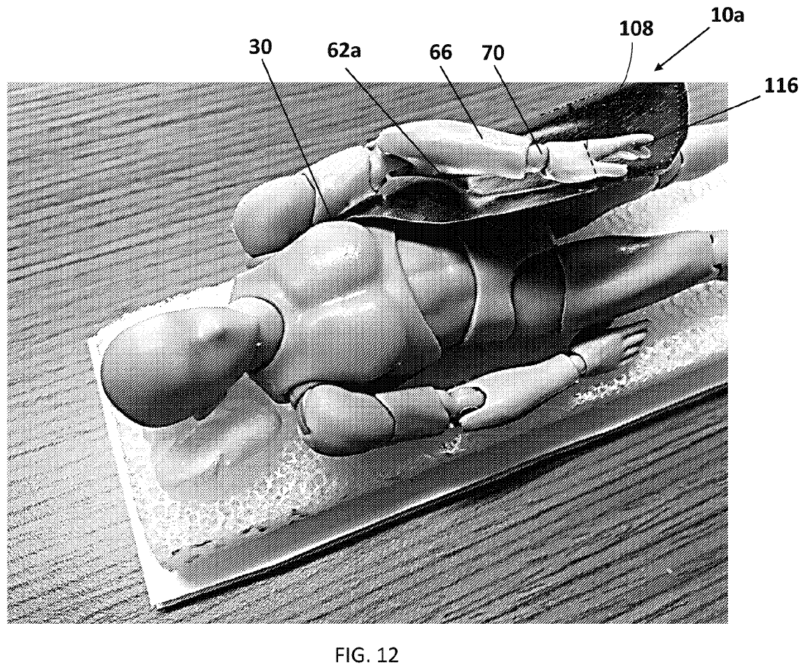

In the configuration depicted in FIGS. 7-10C, support 10a has a first cross-sectional plane 106A, a second cross-sectional plane 106C that is closer to second end 34 than first cross-sectional plane 106A, and a third cross-sectional plane 106B positioned between first cross-sectional plane 106A and second cross-sectional plane 106C. In this configuration, upper portion 26a has a width 108 at third cross-sectional plane 106B that is greater than or equal to a width 110 of upper portion 26a at the first cross-sectional plane 106A and is greater than or equal to a width 114 of upper portion 26a at second cross-sectional plane 106C. The increased width 100 can allow for improved placement of a patient's wrist 70 and provide enhanced stabilization of the patient's arm. For example, the width 108 may be greater than or equal to 110% of length 56 of first lateral portion 54. In the configuration depicted in FIGS. 10A-10C, upper portion 26a has an inflection point 112 at third cross sectional plane 106B. Inflection point 112 may correspond to a valley 116 in distal side 42 that defines support surface 62a.

In the depicted configuration, a radius of curvature is depicted at a first part 120 of unitary body 14. 118 of unitary body 14 at third cross-sectional plane 106B is smaller than a radius of curvature 122 of unitary body 14 at second cross-sectional plane 106C, and the radius of curvature 118 at the third cross-sectional plane 106B is greater than a radius of curvature 126 of unitary body 14 at the first cross-sectional plane 106A to allow for more freedom of movement of a patient's shoulder at first end 30 while providing more support for the patient's forearm 66 and wrist 70 near second end 34.

Support surface 62a may be manipulated by an operator to fit the needs for each specific patient, for example, upper portion 26a may be malleable under sufficient force exerted by an operator or laboratory staff to adjust support surface 62a to a desired position for each TRA based on patient comfort, arm mobility, orthopedic limitations, anatomy, and size of the patient's arm. Once support surface 62a is in the desired position, the patient may rest their arm on support surface 62a without deforming upper portion 26a.

In some configurations, support 10a may comprise a handgrip coupled to upper portion 26a of unitary body 14 and one or more fasteners (not shown) coupled to lower portion 22 configured to attach to upper portion 26a to secure the support 10a in a stationary position. In some configurations, fasteners are disposed on a bottom side of lower portion 22 to secure support 10 by under-mattress anchoring.

In the depicted configuration, lower portion 22 is substantially rigid and upper portion 26a is flexible such that upper portion is moveable when acted upon by a force greater than the typical forces exerted by a patient and/or operator during an intravenous procedure. The flexible upper portion 26a may comprise a composite material (e.g., one or more flexible carbon fiber sheets, other fiber reinforced polymers and/or the like), ballistic nylon, vinyl reinforced with vinyl mesh or any other material that permits the arm support 10a to function as described. For example, the upper portion 26a may be selectively moveable such that a force greater than the weight of a patient's arm is required to deform the flexible material.

In the configurations shown, arm support 10 and 10a are configured to be employed in a cardiac catheterization procedure using a left distal TRA. However, arm support 10 is not limited to these configurations and may be employed for any intravenous procedure where stabilization of a patient's arm may be needed. For example, arm support 10 can be configured for use in a cardiac catheterization procedure using a right distal TRA, where arm support 10 supports the right arm of a patient.

The above specification and examples provide a complete description of the structure and use of illustrative configurations. Although certain configurations have been described above with a certain degree of particularity, or with reference to one or more individual configurations, those skilled in the art could make numerous alterations to the disclosed configurations without departing from the scope of this invention. As such, the various illustrative configurations of the methods and systems are not intended to be limited to the particular forms disclosed. Rather, they include all modifications and alternatives falling within the scope of the claims, and configurations other than the one shown may include some or all of the features of the depicted configurations. For example, elements may be omitted or combined as a unitary structure, connections may be substituted, or both. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one configuration or may relate to several configurations. Accordingly, no single implementation described herein should be construed as limiting and implementations of the disclosure may be suitably combined without departing from the teachings of the disclosure.

The previous description of the disclosed implementations is provided to enable a person skilled in the art to make or use the disclosed implementations. Various modifications to these implementations will be readily apparent to those skilled in the art, and the principles defined herein may be applied to other implementations without departing from the scope of the disclosure. Thus, the present disclosure is not intended to be limited to the implementations shown herein but is to be accorded the widest scope possible consistent with the principles and novel features as defined by the following claims. The claims are not intended to include, and should not be interpreted to include, means-plus- or step-plus-function limitations, unless such a limitation is explicitly recited in a given claim using the phrase(s) “means for” or “step for,” respectively.

1. An arm support for supporting a patient's arm during a cardiac catheterization procedure, the arm support comprising:
a unitary body defining: a lower portion, an upper portion, a first end, a second end, a proximal side, and a distal side;
where the lower portion of the unitary body defines a mount configured to be coupled to a patient support such that the upper portion of the unitary body is supported above at least a part of the patient support; and
where the upper portion of the unitary body defines a support surface extending between the first and second ends, the support surface shaped to support a patient's forearm with the patient's corresponding wrist in either a supinated or pronated position.

2. (canceled)

3. The arm support of claim 1, where the support surface is defined by the proximal side of the unitary body.

4. The arm support of claim 1, where the mount includes an upright portion and a lateral portion, the proximal side of the upright portion faces in a first direction and the lateral portion extending away from the upright portion in the first direction.

5. The arm support of claim 1 where;
the support surface includes an elbow support portion;
where, when the unitary body is coupled to the patient support the elbow support portion of the support surface being lower than a first part of the support surface between the elbow support portion and the first end and lower than a second part of the support surface between the elbow support portion and the second end; and
where the elbow support portion is closer to the first part than to the second part.

6. The arm support of claim 1, where the unitary body comprises a single layer of material spanning the upper and lower portions.

7. (canceled)

8. (canceled)

9. An arm support for supporting a patient's arm during a cardiac catheterization procedure, the arm support comprising:
a unitary body defining: a lower portion, an upper portion, a first end, and a second end;
where the lower portion of the unitary body defines a mount configured to be coupled to a patient support such that the upper portion of the unitary body is supported above at least a part of the patient support; and
an upper portion defining a support surface, the support surface including:
an elbow support portion;
an upper support portion extending from the elbow support portion toward the first end of the unitary body; and
a forearm support portion extending from the elbow support portion toward the second end of the unitary body;
where, when the lower portion is coupled to a patient support the forearm support portion of the support surface extends upward from at least part of the patient support as the distance from the elbow support portion increases.

10. The arm support of claim 9, where the forearm support portion of the surface support has a greater length than the upper support portion.

11. The arm support of claim 9, where the unitary body further defines:
a distal side; and
a proximal side;
where at least a portion of the proximal side is configured to face the patient and at least a portion of the distal side is configured to face away from the patient.

12. The arm support of claim 9, where the upper support portion of the support surface extends downward toward at least part of the patient support with decreasing distance from the elbow support portion.

13. (canceled)

14. The arm support of claim 9, where the upper portion of the unitary body, from bottom to top, sequentially:
extends in a proximal direction over at least a portion of the patient support;
bends toward a distal direction; and
extends in a distal direction such that the support surface is defined by the proximal side of upper portion of the unitary body.

15. The arm support of claim 9, where the support surface includes a wrist support portion extending from the forearm support portion in a direction toward the second end and configured to support a patient's wrist in a supinated or pronated position.

16. An arm support for supporting a patient's arm during a cardiac catheterization procedure, the arm support comprising:
a unitary body defining: a lower portion, an upper portion, a first end, a second end, a proximal side, and a distal side;
where the lower portion of the unitary body defines a mount configured to be coupled to a patient support such that the upper portion of the unitary body is supported above at least a portion of the patient support; and
where the upper portion of the unitary body defines a support surface extending between the first and second ends, the support surface shaped to extend over a portion of the patient such that the distal side of the unitary body is oriented upward to define the support surface to support the patient's arm in either a supinated or pronated position.

17. The arm support of claim 16, where the lower portion of the unitary body is rigid and the upper portion of the unitary body is flexible

18. The arm support of claim 16, where the upper portion comprises flexible carbon fiber sheets, ballistic nylon or vinyl reinforced with vinyl mesh.

19. The arm support of claim 16, having:
a first cross-sectional plane;
a second cross-sectional plane, closer to the second end than the first cross-sectional plane; and
a third cross-sectional plane between the first cross-sectional plane and the second cross-sectional plane;

20. The arm support of claim 16, where:
a radius of curvature of the unitary body at the third cross-sectional plane is smaller than a radius of curvature of the unitary body at the second cross-sectional plane; and
the radius of curvature of the unitary body at the third cross-sectional plane is greater than a radius of curvature of the unitary body at the first cross-sectional plane.

21. The arm support of claim 16, where the upper portion has a greater width at the third cross-sectional plane than at the first or second cross-sectional plane.

22. The arm support of claim 16, where the upper portion of the unitary body has an inflection point at the third cross sectional plane

23. The arm support of claim 16, where a portion of the upper portion extends in substantially the same direction as a portion of the lower portion.

24. The arm support of claim 16, further comprising:
a handgrip coupled to the upper portion of the unitary body; and
fasteners coupled to the lower portion of the unitary body and configured to attach to the upper portion of the unitary body to secure the upper portion in a stationary position.
